(19) United States
(12) Patent Application Publication
(10) Pub. No.: US 2007/0135378 A1
(43) Pub. Date: Jun. 14, 2007

(54) Method for the direct synthesis of oligorhamnosides, composition comprising oligorhamnosides and use thereof as a medicament

(75) Inventors: Jean-Philippe Houlmont, Ramonville (FR); Isabelle Rico-Lattes, Auzielle (FR); Emile Perez, Colomiers (FR); Pascal Bordat, Mervilla (FR)

Correspondence Address: BIRCH STEWART KOLASCH & BIRCH, PO BOX 747, FALLS CHURCH, VA, 22040-0747, US

(73) Assignees: Pierre Fabre Dermo-Cosmetique, Boulogne-Billancourt (FR); Centre National De La Recheche Scientifique, Paris (FR)

(21) Appl. No.: 10/577,654

(22) PCT Filed: Oct. 29, 2004
(86) PCT No.: PCT/FR04/02793

§ 371 Date: May 1, 2006

(30) Foreign Application Priority Data
Oct. 31, 2003 (FR) 0312796

(51) Int. Cl. C12P 19/04; A61K 31/715

(52) U.S. Cl. 514/61; 536/123

(57) ABSTRACT

The invention relates to a method of preparing oligorhamnosides, comprising a one-pot reaction in acetonitrile without any rhamnose protection or deprotection reaction. The invention also relates to a composition a mixture of oligorhamnosides, which can be obtained using said method and which has between 2 and 12 rhamnose units. The invention further relates to a medicament comprising said composition, which is preferably intended to regulate inflammatory mechanisms, and to a cosmetic treatment method.

The present invention relates to a method for the direct synthesis of oligorhamnosides. The synthesis method consists of a one-pot reaction in acetonitrile, without any rhamnose protection or deprotection reaction. The oligorhamnoside mixture obtained exhibits anti-inflammatory activity.

The inflammatory reaction is a response by the immune system of an organism faced with an attack against its cells or vascularized tissues by a pathogen such as a virus or a bacterium, or by a chemical or physical attack. Often painful, inflammation is generally a healing response. In certain cases, however, (rheumatoid arthritis, Crohn's disease, autoimmune diseases, etc.) it can have consequences more serious than the original stimulus.

Contact hypersensitivity reactions correspond to specific immunity reactions directed against antigens located on cells or in tissues, at the origin of cellular lesions or inflammatory reactions. These hypersensitivity reactions can develop within the framework of defense mechanisms with respect to a pathogenic microorganism or in the case of allergic reactions. They utilize various types of cells, in particular skin cells and certain leucocytes, not to mention endothelial cells whose role is preponderant in inflammatory reactions.

The intercellular interactions which intervene generally imply specific recognition phenomena between ligands and receptors. During the past twenty years, many cellular surface receptors have been identified, such as proteins capable of ensuring specific recognition with certain sugars such as fucose and rhamnose.

Lectins are proteins imbedded in the membranes of eukaryotic cells which play a very important role in adhesion and recognition phenomena between cells, in particular during inflammatory processes. Membrane lectins are implicated in particular in endocytosis, intracellular transport of glycoconjugates and endothelial permeability. Moreover, these proteins, often transmembrane proteins, contribute to specific antigen recognition (extracellular domain) and to cell activation (intracellular domain). Lectins can specifically recognize certain sugars, in particular rhamnose.

The study and therapeutic use of oligorhamnosides more than ever requires the availability of these products in great quantities. Unfortunately, they are difficult to isolate in a homogeneous form from living cells, due to the fact that they exist in the form of microheterogeneous mixtures. The purification of such compounds, when possible, is difficult and generally provides a very poor yield. These constraints demonstrate the interest in having a simple and effective oligorhamnoside synthesis method.

During the last century, a number of researchers have attempted to provide original oligosaccharide synthesis methods. The first were initiated by Fischer, Köenigs and Knorr, Lemieux (Lemieux, R. U., Morgan, A. R., The preparation and configurations of tri-O-acetyl-alpha-D-glucopyranose 1,2-(orthoesters). Canadian journal of chemistry, 1965. 43: p. 2199-2204) which introduced the concepts of activation and protection of saccharide units. Next, Paulsen (Paulsen, H., Kutschker, W., Lockhoff, O., Building units for Oligosaccharides, XXXIII: synthesis of beta-glycosidically linked disaccharides of L-rhannose. Chemical Berstein, 1981. 114: p. 3233-3241), and more recently Schmidt and Seeberger (Seeberger, P.H.H.W.C, Solid-phase oligosaccharide synthesis and combinatorial carbohydrate libraries. Chemical reviews, 2000. 100 (no. 12): p. 4349-4393; Seeberger, P. H., Plant, O. J., Synthesis of oligosaccharides, reagents and methods related thereto. 2001, MIT (Cambridge Mass.): United States, p. 50) proposed more complex methods on solid supports in order to improve reaction yields and specificities. The synthesis of oligosaccharides had as an objective the exploitation of their biocompatibility in order to discover new therapeutic substances. However, these methods are based on strategies which require all of the sugar protection and deprotection steps. These synthesis methods thus remain unwieldy and are, unfortunately, accompanied by very low yields which decrease the potential for industrial production. It is thus of primary importance to find a suitable original method which would allow the synthesis of these compounds with a minimum of reaction steps.

In a surprising way, the inventors have discovered that it is possible to synthesize oligorhamnosides directly in acetonitrile (one-pot synthesis) without any rhamnose protection or deprotection reaction.

This synthesis in a single-step reaction uses the specific solubility properties of rhamnose in acetonitrile: rhamnose is extremely insoluble in cold acetonitrile and only slightly more soluble in hot acetonitrile.

Within the meaning of the present invention, “oligorhamnoside” is understood to mean any oligomer comprised of rhamnose motifs, of levorotatory or dextrorotatory configuration, advantageously levorotatory, linked together by glycosidic bonds of α or β configuration. The aforementioned oligomer can be in linear or branched form.

The present invention relates to an oligorhamnoside preparation method comprising the following successive steps:
a) self-condensation of rhamnose in a single reaction step in acetonitrile in the presence of an acid catalyst and precipitation of the oligorhamnosides thus formed; then
b) recovery by filtration of the precipitate obtained following step a) comprising the oligorhamnosides.

The originality of the method is due to the fact that the rhamnose self-condensation reaction in acetonitrile is performed directly without a preliminary rhamnose hydroxyl-function protection step and, similarly, without a successive rhamnose hydroxyl-function deprotection step.

During the aforementioned step a), the work takes place advantageously in a homogeneous medium to limit excess rhamnose in the final crude product, while working at saturation to ensure the rapid precipitation of the oligorhamnosides. Thus, the acetonitrile solution is advantageously saturated with rhamnose.

The mixture of rhamnose, acetonitrile and acid catalyst is advantageously brought into reaction under heating, and possibly under stirring, at a temperature between 20° C. and 120° C., even more advantageously between 35° C. and 75° C. The self-condensation temperature should not exceed 120° C. in order to avoid degradation of the sugars. In an advantageous manner, the heating temperature is approximately 65° C. at atmospheric pressure. The mixture of rhamnose, acetonitrile and acid catalyst is advantageously mixed for between 5 minutes and 24 hours, still more advantageously for 3 hours.

In the chemistry of sugars, it is highly classical to use an acid catalyst to favor the formation of glycosidic bonds. Within the framework of the present invention, the acid catalyst is advantageously chosen from the group comprised of hydrochloric acid, sulfuric acid, phosphoric acid, ortho-, meta- and para-toluenesulfonic acid, benzene-sulphonic acid, substituted benzene-sulphonic acids, methane-sulphonic acid, Lewis acids, in particular zinc chloride and ferric chloride, clay acids, in particular montmorillonite K-10, synthetic resin acids, zeolites and combinations thereof.

The substituted benzene-sulphonic acids are advantageously the ortho-, meta- and para-bromobenzenesulfonic acids.

The Lewis acids are, for example, zinc chloride, ferric chloride or any other metal, metalloid or lanthanide halide.

As examples of synthetic resin acids, the types “Amberlyst®”, “Amberlite®” and “Dowex®” can be cited in particular.

The quantity of catalyst must be controlled because the reaction is very rapid. In order to further control the reaction, it is necessary to slow it. The quantity of acid catalyst added is advantageously fixed. The quantity of catalyst added corresponds advantageously to approximately 0.1 mole of catalyst for 1 mole of rhamnose.

The water formed during the self-condensation reaction of step a) is advantageously eliminated, physically or chemically. As an example of a physical technique for eliminating the water formed during the synthesis, distillation or the use of an adsorbent can be cited in particular. As an example of a chemical technique for eliminating the water formed during the synthesis, the use of a desiccation agent can be cited in particular.

According to an advantageous variant of the invention, the water formed during the self-condensation reaction of step a) is eliminated by means of a desiccation agent chosen from the group comprised of the carbonates, the sulfates, calcium chloride, phosphorus pentoxide, the molecular sieves or combinations of these various desiccation agents. The desiccation agent can be introduced directly into the reaction medium or be present at the level of the solvent vapors inside a “Soxhlet-type” solid/liquid extraction cartridge.

According to a variant of the invention, the self-condensation reaction of step a) is carried out at atmospheric pressure under an atmosphere of inert gas, such as argon or nitrogen.

According to another variant of the invention, the self-condensation reaction of step a) is carried out at reduced pressure (preferably approximately 260 mbar), advantageously in an autoclave.

The reagents can be introduced in a fractional or continuous way in order for the reaction to always be carried out in the most favorable molar ratios.

Prior to step b), the reaction mixture is advantageously cooled to a temperature ranging between the condensation reaction temperature and 0° C., still more advantageously to ambient temperature, that is to say to approximately 20° C.

This additional cooling step favors the precipitation of the oligorhamnosides formed during step a).

The precipitate recovered during step b) is called precipitated (P1). It is advantageously recovered by filtration such as Buchner filtration.

The aforementioned precipitate (P1) is advantageously washed in acetonitrile. The filtrate obtained following step b) is evaporated under reduced pressure in order to recover a second precipitate ((P2)) containing unreacted rhamnose but also the rhamnosylated derivatives of precipitate (P1) passed into the solution.

The precipitation of oligorhamnosides during the reaction releases molecules of the solvent (acetonitrile). Thus is produced in a reversible manner a solubilization of the lowest masses, and this is why the precipitate (P2) obtained by evaporation of the acetonitrile phase contains a rich part of rhamnose but also a fraction of the synthesized oligorhamnosides.

The oligorhamnosides obtained exhibit a maximum degree of polymerization equal to 12, advantageously ranging between 2 and 9.

It is thought that the modification of the operating conditions does not contribute to the change in the distribution of the masses but only to a change in the oligorhamnoside formation kinetics. Indeed, if it is considered that starting from a certain size, an oligomer is no longer soluble in acetonitrile and precipitates, then this phenomenon can be accompanied by two other underlying and opposite phenomena. The first is the release of solvent molecules which will then be available to further solubilize the smallest oligomers, and the second is a coprecipitation phenomenon. An oligomer of sufficiently large mass can cause the precipitation of its smaller homologues when it becomes insoluble in the reaction medium.

These three phenomena, precipitation, coprecipitation and resolubilization, are probably at the origin of qualitative uniformity in the distribution of mass.

The rhamnose motifs have up to three of their hydroxyl functions implicated in the formation of glycosidic bonds. The rhamnose motif having four hydroxyl functions, it could in theory form four glycosidic bonds. However, during the implementation of the method according to the invention, it is noted that at most three of the rhamnose motif hydroxyl functions are implicated in the formation of glycosidic bonds. It is thought that this is due to problems of steric obstruction.

The oligorhamnoside yield by weight of the method according to the invention lies between 30 and 60% with respect to the quantity by weight of rhamnose introduced.

The present invention also relates to a composition comprised of a mixture of oligorhamnosides likely to be obtained by the method according to the invention, the aforementioned oligorhamnosides containing from 2 to 12 rhamnose motifs, advantageously from 2 to 9 rhamnose motifs.

The distribution of oligorhamnosides as a function of their degree of polymerization follows roughly a Poisson distribution.

The rhamnose motifs have up to three of their hydroxyl functions implicated in the formation of glycosidic bonds. The glycosidic bonds can be α or β bonds.

The present invention also relates to a medicament comprised of a composition according to the invention, that is to say an oligorhamnoside mixture such as defined previously.

The medicament according to the invention is advantageously intended to regulate inflammatory mechanisms.

The medicament is in particular intended for the prevention or treatment of allergic, inflammatory or immune reactions or pathologies of the skin and/or mucous membranes. The medicament according to the invention is also intended to inhibit the immune response related to inflammatory stress.

The medicament according to the invention is in particular intended to inhibit the activation of leucocytes, such as human granulocytes, in particular human neutrophils and mast cells which prevent the release of the preformed mediators of the immune reaction. It also makes possible inhibition of the adhesion of circulating lymphocytes and endothelial cells, thus preventing the transmigration of these leucocytes to the inflammation site. It also makes possible inhibition of the secretion of keratinocytic cytokines, activators of T lymphocytes and Langerhans cells such as IL-1 and TNF-α, or of adhesion molecules such as ICAM-1 and VCAM, which contribute to the recruitment and trans-endothelial passage of leucocytes. The medicament according to the invention is also an inhibitor of the keratinocytic hyperplasia phenomenon.

The medicament according to the invention is also an inhibitor of antigen processing by the dendritic cells of the skin, of maturation of antigen-presenting cells, namely dermal dendritic cells and Langerhans cells, and of the recognition phenomenon between lymphocytes and antigen-presenting cells.

Thus, the medicament according to the invention is intended for the prevention or treatment of diseases chosen from the group comprised of atopic and/or contact eczema, inflammatory dermatoses, irritant dermatitis, acne, autoimmune diseases such as psoriasis, photo-immunosuppression, vitiligo, pityriasis, sclerodermas, rheumatoid arthritis, Crohn's disease and graft rejection.

The medicament according to the invention is also intended for the prevention and treatment of age-related chronic inflammatory problems and their consequences. The medicament is in particular intended for the prevention or treatment of diseases chosen from the group comprised of anaphylactic sensitivities, pigmentary anomalies of the skin, dermal hypervascularity and inflammatory fissuring.

According to a variant of the invention, the medicament is intended to reduce the allergenic and/or irritant character of a composition or perfume.

The medicament according to the invention advantageously contains from 0.001% to 50% by weight of oligorhamnosides.

The medicament according to the present invention can be formulated for administration by any route. It is advantageously formulated to be administered by topical, oral, subcutaneous, injectable, rectal and vaginal routes.

When the medicament is formulated to be administered by oral route, the aforementioned medicament can appear in the form of an aqueous solution, an emulsion, tablets, gelatin capsules, capsules, powders, granules, solutions or oral suspensions.

When the medicament is formulated to be administered by subcutaneous route, the aforementioned medicament or the aforementioned composition can appear in the form of sterile injectable ampules.

When the medicament is formulated to be administered by rectal route, the aforementioned medicament can appear in the form of suppositories.

When the medicament is formulated to be administered by vaginal route, the aforementioned medicament can appear in the form of vaginal suppositories.

The medicament according to the invention is preferably a topical application. Thus, the medicament can be formulated so as to appear, for example, in the form of an aqueous solution, a white or colored cream, a pomade, a milk, a lotion, a gel, an ointment, a serum, a paste, a foam, an aerosol or a stick.

The quantity of the medicament according to the invention to be administered depends on the gravity and age of the ailment treated. Naturally, the doctor will also adapt the dosage according to the patient.

The present invention also relates to a method for the cosmetic treatment of skin and/or mucous membranes that are sensitive, irritated, intolerant, of an allergic tendency, aged, exhibiting danger signs, exhibiting a disorder of the cutaneous barrier, exhibiting cutaneous redness or exhibiting a non-pathological immunological imbalance related to intrinsic, extrinsic or hormonal aging, wherein it consists of applying to the skin and/or the mucous membranes a composition comprised of a mixture of oligorhamnosides such as previously defined.

The present invention also relates to a cosmetic treatment method to slow the natural aging of the skin and/or to prevent the accelerated aging of skin subjected to external attacks, in particular to prevent photo-induced aging of the skin, wherein it consists of applying to the skin and/or the mucous membranes a composition comprised of a mixture of oligorhamnosides such as previously defined.

Within the framework of a cosmetic use, the composition according to the invention advantageously contains from 0.001% to 50% by weight of oligorhamnosides with respect to the total weight of the composition.

When the cosmetic composition is formulated to be administered by topical route, the aforementioned composition can appear, for example, in the form of an aqueous solution, a white or colored cream, a pomade, a milk, a lotion, a gel, an ointment, a serum, a paste, a foam, an aerosol, a shampoo or a stick.

Other characteristics and advantages of the invention appear in the continuation of the description with the examples presented below. The following figures will be referred to in these examples. These figures and examples are intended to illustrate the present invention and cannot in any case be interpreted as limiting its scope.

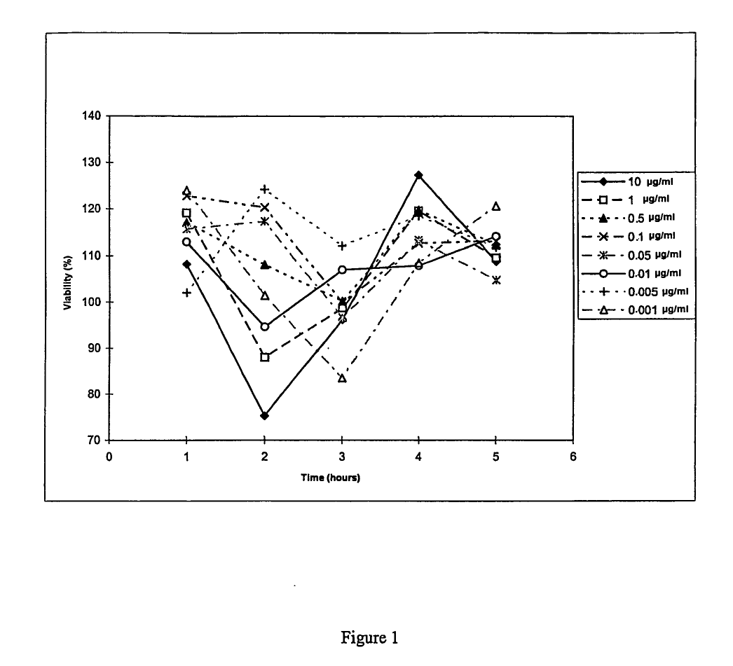

FIG. 1: Viability of endothelial cells arising from peripheral lymphatic ganglia in the presence of rhamnose.

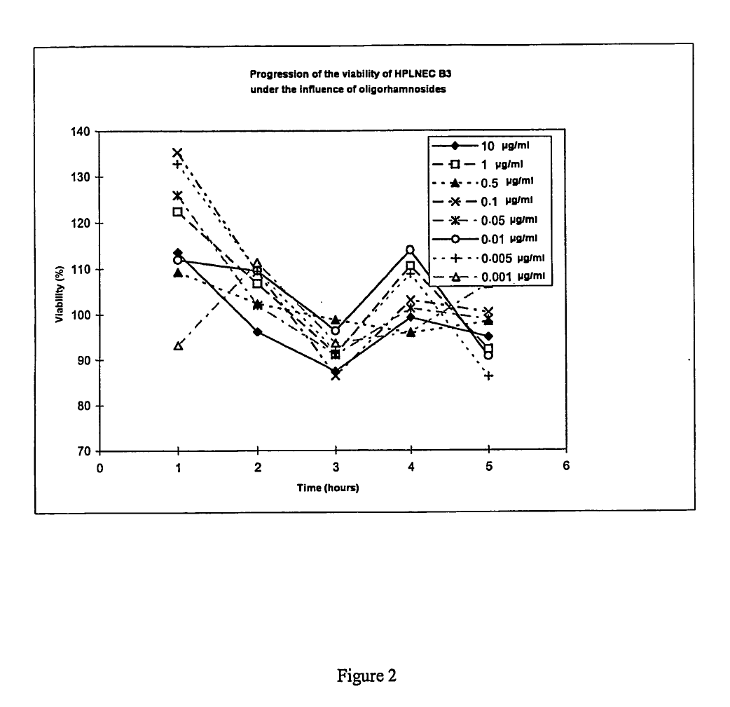

FIG. 2: Viability of endothelial cells arising from peripheral lymphatic ganglia in the presence of oligorhamnosides.

### EXAMPLE 1

### Direct Synthesis of Oligorhamnosides

Into a 100 ml two-neck round-bottom flask, surmounted with a condenser equipped with a desiccant (CaCl₂) trap, 25 ml of acetonitrile (dried over molecular sieve 3 Å) is introduced under argon. Rhamnose (360 mg) is introduced into hot (65° C.) acetonitrile, in four equivalent fractions, while waiting for complete dissolution before adding the subsequent fraction.

0.3 ml of a 0.6 M solution (weight ratio of 0.1 g/g of rhamnose) of the acid catalyst p-toluenesulfonic acid (PTSA) is added in the mixture maintained under argon. The solution is stirred (magnetic stirrer) at 65° C. for 40 minutes.

After the reaction, stirring is stopped and the mixture cooled at ambient temperature for 30 minutes.

The precipitate (P1) formed is recovered by filtration under vacuum on sintered glass of porosity 4, then is washed in acetonitrile and in anhydrous ether, then taken up in a minimum of water and lyophilized. Thus 126 mg of a white powder corresponding to the oligorhamnosides is obtained, which is a yield of 35%.

The recovered filtrate is evaporated in a rotary evaporator under reduced pressure (15 mmHg), and thus 234 mg of the solid (P2) corresponding to a rhamnose/oligorhamnoside mixture of low molecular weight is obtained. The size distribution and the masses of the synthesized oligosaccharides are measured by HPLC and mass spectrometry, respectively.

The oligorhamnosides (P1) and (P2) are analyzed by high-performance liquid chromatography (HPLC) on a Phenomex REZEX RSO-oligosaccharide column (200×10.0 mm) provided with a guard column (60×10.0 mm). The eluent is 100% H₂O at a flow rate of 0.3 ml/min and at a column temperature of 80° C. The injected samples, 10 μl at a concentration of 50 mg/ml in water, are detected using a differential refractometer. Under these conditions, the analysis lasts 50 minutes and makes it possible to separate the rhamnose (retention time of 47 minutes) from the rhamnosylated derivatives (retention time of 15 to 44 minutes).

The masses of the oligorhamnosides (P1) and (P2) are determined by electrospray mass spectrometry for degrees of polymerization of 1 to 5 and LSIMS for degrees of polymerization of 5 to 12. These analyses of mass reveal a maximum degree of polymerization equal to 12. All glycosidic bond types are found, except the rhamnose motif where all the hydroxyl functions have reacted.

### EXAMPLE 2

### Direct Synthesis of Oligorhamnosides

This method is according to the protocol of example 1, but in this case the solution is stirred at 40° C. for a duration of 40 minutes as according to example 1.

After the reaction and treatments according to the same protocols as those described in example 1, 72 mg of precipitate (P1) is obtained, which is a yield of 20%.

The weight of the solid (P2) recovered in this example is 288 mg.

HPLC and mass spectrometry analyses lead to the same results as those obtained according to the protocol of example 1.

### EXAMPLE 3

### Direct Synthesis of Oligorhamnosides

This method is according to the protocols of examples 1 and 2, but in this case the solution is stirred at 65° C., but for a duration of 6 hours. After the reaction and treatments according to the same protocols as those described in examples 1 and 2, 144 mg of brownish-yellow precipitate (P1) is obtained, which is a yield of 40%.

The weight of the solid (P2) recovered in this example is 216 mg.

HPLC and mass spectrometry analyses lead to the same results as those obtained according to the protocols of examples 1 and 2.

### EXAMPLE 4

### Direct Synthesis of Oligorhamnosides

This method is according to the protocols of examples 1, 2, 3, but in this case the solution is stirred at 40° C. for a duration of 6 hours. After the reaction and treatments according to the same protocols as those described in examples 1 to 3, 126 mg of precipitate (P1) is obtained, which is a yield of 35%.

The weight of the solid (P2) recovered in this example is 234 mg.

HPLC and mass spectrometry analyses lead to the same results as those obtained according to the protocols of examples 1 to 3.

### EXAMPLE 5

### Direct Synthesis of Oligorhamnosides

This method is according to the protocol of example 1, but in this case the total quantity of rhamnose introduced into 65° C. acetonitrile is 200 mg.

After the reaction and treatments according to the same protocols as those described in examples 1 to 4, 30 mg of precipitate (P1) is obtained, which is a yield of 15%.

The weight of the solid (P2) recovered in this example is 170 mg.

HPLC and mass spectrometry analyses lead to the same results as those obtained according to the protocols of examples 1 to 4.

### EXAMPLE 6

### Direct Synthesis of Oligorhamnosides

This method is according to the protocol of example 1, but in this case the quantity of the catalyst PTSA is higher, with a weight ratio of 0.2 g/g of rhamnose.

After the reaction and treatments according to the same protocols as those described in examples 1 to 5, 126 mg of precipitate (P1) is obtained, which is a yield of 35%.

The weight of the solid (P2) recovered in this example is 234 mg.

HPLC and mass spectrometry analyses lead to the same results as those obtained according to the protocols of examples 1 to 5.

### EXAMPLE 7

### Direct Synthesis of Oligorhamnosides

This method is according to the protocol of example 1, but with a higher volume of acetonitrile and weight of rhamnose and for a longer duration.

Into a 100 ml two-neck round-bottom flask, surmounted with a condenser equipped with a desiccant (CaCl₂) trap, 80 ml of dry acetonitrile is introduced under argon. Rhamnose (550 mg) is introduced into hot (65° C.) acetonitrile in small fractions. In this case the solution is stirred at 65° C. for a duration of 4 hours.

After the reaction and treatments according to the same protocols as those described in examples 1 to 6, 137.5 mg of precipitate (P1) is obtained, which is a yield of 25%.

The weight of the solid (P2) recovered in this example is 412.5 mg.

HPLC and mass spectrometry analyses lead to the same results as those obtained according to the protocols of examples 1 to 6.

### EXAMPLE 8

### Direct Synthesis of Oligorhamnosides

This method is according to the protocol of example 1, but with a resin acid such as Amberlyst® 15 dry replacing the p-toluenesulphonic acid (PTSA).

This catalyst (500 mg) is added to the mixture maintained under argon.

After the reaction and treatments according to the same protocols as those described in examples 1 to 7, 90 mg of precipitate (P1) is obtained, which is a yield of 25%.

The weight of the solid (P2) recovered in this example is 270 mg.

HPLC and mass spectrometry analyses lead to the same results as those obtained according to the protocols of examples 1 to 7.

### EXAMPLE 9

### Direct Synthesis of Oligorhamnosides

A 100 ml two-neck round-bottom flask is surmounted by a Soxhlet-type solid/liquid extractor equipped with an extraction cartridge, itself surmounted with a condenser equipped with a vacuum inlet. The extraction cartridge is filled with dry calcium chloride in a CaCl2/rhamnose weight ratio of 20 and surmounted with glass wool.

80 ml of acetonitrile (dried over molecular sieve 3 Å) is introduced into the flask under argon.

Rhamnose (550 mg) is introduced into hot (65° C.) acetonitrile, in four equivalent fractions, while waiting for complete dissolution before adding the subsequent fraction.

1 ml of a 0.6 M solution (weight ratio of 0.2 g/g of rhamnose) of the acid catalyst p-toluenesulfonic acid (PTSA) is added in the mixture maintained under argon.

The assembly is then connected to a vacuum source (filter pump) equipped with a pressure regulator in order to reach 260 mbar. Once this pressure is reached, the solution is stirred (magnetic stirrer) at 65° C., which is the temperature corresponding to the reflux of acetonitrile at the chosen pressure. The vacuum and the temperature are maintained for 4 hours, which corresponds to several filling/siphoning cycles of the extractor.

After the reaction, the vacuum and stirring are stopped, and the mixture cooled at ambient temperature for 30 minutes.

After the reaction and treatments according to the same protocols as those described in examples 1 to 8, 275 mg of precipitate (P1) is obtained, which is a yield of 50%.

The weight of the solid (P2) recovered in this example is 275 mg.

HPLC and mass spectrometry analyses lead to the same results as those obtained according to the protocols of examples 1 to 8.

### EXAMPLE 10

### Pharmacological Analysis of Oligorhamnosides

The various immune cells acting in these inflammation processes were studied. They are the dendritic cells of the skin, the endothelial cells, certain leucocytes and the keratinocytes.

1) Principles of Cellular Viability Measurement Techniques
MTT [3-(4,5-dimethyldiazol-2-yl)-2,5-diphenyl tetrazolium bromide] reduction technique (sold by Sigma).

This technique corresponds to a calorimetric test allowing quantification of living, metabolically active cells in a non-radioactive manner. MTT is a cationic molecule which is bound to the membranes of mitochondria in a potential-dependant fashion. On the level of the mitochondria, MTT will be reduced to formazan blue by mitochondrial dehydrogenase. The living cells are thus colored blue, in contrast with the dead cells which remain transparent. The measure of viability is then carried out by measurement of the optical density using an automatic reader.

This method of analysis, however, seems to be better adapted for adherent cells (keratinocyte-type) than for non-adherent cells (monocytes and dendritic cells). Another study was thus envisaged to conclude on the cytotoxicity of the oligorhamnosides with respect to the differentiated cells analyzed, namely flow cytometry in the presence of propidium iodide.
XTT tetrazolium salt reduction technique.

This is a technique allowing quantification of cellular proliferation and of the number of living (metabolically active) cells, without the incorporation of radioactive isotopes. XTT, yellow in color, is a cationic molecule which is bound to the membranes of mitochondria in a potential-dependant fashion, as does MTT.

On the level of the mitochondria, XTT will be reduced to formazan (orange) by mitochondrial tetrazolium reductase. This method, more costly than the MTT method, does not require in its protocol the lysis of cells by SDS to release the dye. Indeed, the reduction product is soluble within the cell. The method is thus faster. Living cells, in the absence and presence of a treatment become colored, in contrast with dead cells which remain colorless. The level of formazan product is detected with the spectrophotometer at a wavelength of 450 nm and is directly proportional to the number of metabolically active cells.

2) Toxicity Tests
Keratinocytes were isolated and placed in culture from human skin biopsies. Measurements of optical density (absorbance) of the 4 wells treated with the same product concentration were averaged. This average was compared with the average of the measurements obtained for the 4 control wells (Student's t-test—comparison of means—significant difference at 95% if p<0.05 and 99% if p<0.01).

The viabilities of the treated cells are expressed as a percentage compared to the control (untreated cells) of 100% (OD treated/OD control×100).

Rhamnose does not exhibit cytotoxicity (see Table 1), even for the highest concentrations.

TABLE 1
Viability of keratinocytes in the presence
of various rhamnose concentrations.

| | | Rhamnose | Rhamnose | Rhamnose | Rhamnose |
|---|---|---|---|---|---|
| | | 1 | 0.1 | 0.01 | 0.001 |
| | Control | mg/ml | mg/ml | mg/ml | mg/ml |
| % viability | 100 | 104 | 98 | 100 | 95 |
| p (Student) | | 0.504 | 0.679 | 0.991 | 0.407 |

The oligorhamnosides exhibit toxicity at high concentrations, those above 5 mg/ml (see Table 2).

TABLE 2
Viability of keratinocytes in the presence of various oligorhamnoside concentrations.

| | | Oligorhamnosides | Oligorhamnosides | Oligorhamnosides | Oligorhamnosides | Oligorhamnosides |
|---|---|---|---|---|---|---|
| | Control | 5 mg/ml | 2 mg/ml | 1 mg/ml | 0.1 mg/ml | 0.1 mg/ml |
| % viability | 100 | 75 | 92 | 92 | 102 | 117 |
| p (Student) | | <0.01 | 0.072 | 0.104 | 0.554 | <0.01 |

Endothelial cells were placed in culture, immortalized and stabilized in their phenotype. The cell lines studied were appendix endothelial cells, brain microvascular endothelial cells, mesenteric lymphatic ganglia endothelial cells, peripheral lymphatic ganglia endothelial cells and skin microvascular endothelial cells.

The cytotoxicity test was carried out by means of a biochemical test on the transformation of a tetrazolium salt, MTT. The results obtained are very positive, and no toxicity is demonstrated with pentyl-rhamnoside (see FIGS. 1 and 2). Viability is indeed always greater than 85%, and this is true for all of the cells lines studied.

FIG. 1: Viability of endothelial cells arising from peripheral lymphatic ganglia in the presence of rhamnose.

FIG. 2: Viability of endothelial cells arising from peripheral lymphatic ganglia in the presence of oligorhamnosides.

Noted in particular is the appearance of a stimulation peak corresponding to 4 hours of incubation, which is the time necessary for the initiation of protein synthesis. The presence of this peak is interesting because it indicates that the cells tolerate the oligorhamnosides (absence of toxicity) and assimilate them. These products appear to enrich the culture medium.

These results are similar for the other endothelial cell lines.

3) Influence of Alkyl-Rhamnosides on Human Cells Cultivated in a Pro-Inflammatory Medium

On Dendritic Cells

Three groups of cells are analyzed: 1) those which have not been exposed to oligorhamnosides or the selected activation signals, which will be used as negative controls, 2) those which were incubated for 24 hours with the activation signals, which will be used as positive controls, and 3) those which had been in contact with the oligorhamnosides for 24 hours.

The culture medium used is a classical medium of type RPMI 1640 (Bioproducts), supplemented with 10% FCS, glutamine (2 mM) and the antibiotics penicillin and streptomycin (Bioproducts). The rinse liquid used is a PI buffer (Bioproducts). The cellular activator used is INFγ (SIGMA).

The results are given in Table 3 below:

TABLE 3

| | DMSO control | | LPS control | | Null control | |
|---|---|---|---|---|---|---|
| Viability | 1.27% | | 85.63% | | 92.6% | |
| (propidium | | | | | | |
| iodide, PI) | | | | | | |
| CD86 B7.2 | 0.17% | | 49.96% | | 7.4% | |
| (Maturation) | | | | | | |
| | 10 | 5 | 1 | 0.5 | 0.1 | 0.05 |
| | mg/ml | mg/ml | mg/ml | mg/ml | mg/ml | mg/ml |
| Viability | 90% | 91% | 91% | 92% | 93% | 93% |
| (propidium | | | | | | |
| iodide, PI) | | | | | | |
| CD86 B7.2 | 10% | 6% | 4% | 4% | 5% | 3% |
| (Maturation) | | | | | | |

Viability is determined from the mortality translated directly by the proportion of PI signal. The results presented in Table 3 reflect cellular viability (100-mortality), the proportion of living cells in each well studied.

The allergenic effect is reflected by the maturation of the dendritic cells and the expression of the CD86 B7.2 receptor. The more this receptor is expressed, the higher the proportion of mature cells and the more the rhamnosylated derivative studied has an allergenic effect.

The oligorhamnosides cause neither cell death nor activation or accelerated maturation of the dendritic cells. Indeed, the dendritic cells studied continue to express the specific receptors of the immature dendritic cells or do not exhibit an increase in the expression of the other control membrane markers for maturation. In particular, the results are negative with regard to the high density of CD86 molecules characteristic of mature dendritic cells, and thus the dendritic cells did not regard the oligorhamnosides tested as activation signals capable of accelerating or transforming an immature DC into an activated or mature DC.

Assay of IL-8 Released by Keratinocytes Stimulated with Activator IL-1β

The assay of IL-8 makes it possible to evaluate the anti-inflammatory properties of oligorhamnosides when they are added simultaneously with the activator used, IL-1β. This assay is carried out on the supernatant recovered after the treatment. Indeed, the normal human keratinocytes (NHK) placed in contact with the activator IL-1β secretes IL-8 and its concentration in the cellular supernatants passes from 3.2 pg/ml/μg proteins for untreated NHK to approximately 32 pg/ml/μg proteins for those which are activated.

To refine the cytokine assay, an additional assay is carried out, that of cellular proteins. It will make it possible to express, for each well, the quantity of IL-8 secreted per μg of cellular protein (BCA method described in the experimental section).

The keratinocytes are treated for 24 hours with the oligorhamnosides and the agonist IL-1β. After 24 hours, the cytokine IL-8 released in the cellular supernatants is quantified by means of an ELISA (enzyme-linked immunosorbent assay).

The standard range of the commercial kit makes it possible to relate the absorbance measured at 450 nm and the concentration in IL-8 (pg/ml) present in each of the cellular supernatants.

The various concentrations tested are evaluated on 4 wells of cells. Thus 4 distinct IL-8 concentration values (pg/ml) are obtained for each treatment condition. These quantifications can be reported as quantity of proteins per well.

A first experiment consists of incubating the IL-1β and the oligorhamnosides together for 24 hours. The results below (see Table 4) represent the means of these 4 values and the standard deviation.

TABLE 4
Results of treatment of NHK with IL-1β and various
oligorhamnoside concentrations for 24 hours.

| | Mean IL-8 | |
|---|---|---|
| Treatment | (pg/ml/μg proteins) | % inhibition |
| Negative control | 3.6 ± 0.9 | |
| Positive control (IL-1β) | 32.3 ± 8.7 | |

IL-1β stimulation, 1 ng/ml

| | | |
|---|---|---|
| 1 mg/ml | 23.5 ± 6.6 | 27% |
| 0.1 mg/ml | 31.7 ± 5.5 | / |
| 0.01 mg/ml | 31 ± 9.3 | / |
| 0.001 mg/ml | 30.1 ± 5.9 | / |

The results demonstrate that the placement of oligorhamnosides in direct contact with the activator contributes to its inhibition (27%, 1 mg/ml) (see Table 5), and that this effect is no longer detected when the oligorhamnoside concentration decreases.

A second experiment is carried out in which the oligorhamnosides are used in pretreatment for 8 hours on the NHK before adding the IL-1β and the oligorhamnosides together for 24 hours.

As previously, the two assays are carried out (IL-8 and proteins). The results are summarized in the following table (see Table 5).

TABLE 5
Results of treatment of NHK with IL-1β and various
oligorhamnoside concentrations for 24 hours after 8
hours of pretreatment with oligorhamnosides.

| | Mean IL-8 | |
|---|---|---|
| Treatment | (pg/ml/μg proteins) | % inhibition |
| Negative control | 5.5 ± 0.9 | |
| Positive control (IL-1β) | 141.0 ± 12.7 | |

IL-1β stimulation, 1 ng/ml

| | | |
|---|---|---|
| 1 mg/ml | 101.3 ± 16 | 28% |
| 0.1 mg/ml | 117.0 ± 19.8 | 17% |
| 0.001 mg/ml | 116.9 ± 15 | 17% |

The results show that the pretreatment placing in contact of NHK with oligorhamnosides does not truly contribute to an increase in inhibition (28%, 1 mg/ml). However, this inhibition is now detectable at weaker concentrations. This makes it possible to conclude that pretreatment with oligorhamnosides favors inhibition of the release of IL-8 in the supernatant.

It can be supposed that the oligorhamnosides occupy the reaction sites of the keratinocytes leading to inhibition of the secondary reaction (blocking of the IL-1β sites), however we cannot conclude on the inflammatory process inhibition mechanism.

Assay of Prostaglandin (6-ketoPGF_{1α}) After Stimulation of Keratinocytes by PMA.

Prostaglandin I2 (PGI₂) is an unstable metabolite of the arachidonic acid degradation pathway. Like prostaglandin E2, it is a mediator of inflammation with vasodilatory properties. The instability of PGI₂ lies in its rapid conversion (non-enzymatic hydration) to 6-keto prostaglandin F_{1α} (6-ketoPG_{1α}). It is thus this prostaglandin which is measured as a marker for PGI₂ synthesis.

TABLE 6
Results of treatment of NHK with IL-1β, PMA and
various oligorhamnoside concentrations after 4 hours
of pretreatment with oligorhamnosides.

| | Mean 6-ketoPGF_{1α} | |
|---|---|---|
| Treatment | (pg/ml/μg proteins) | % inhibition |
| Negative control | 24.4 ± 5.5 | |
| Positive control | 27.3 ± 2.2 | |
| (1 ng/ml IL-1β) | | |
| Positive control | 33.5 ± 2.3 | |
| (10 ng/ml PMA) | | |

IL-1β stimulation, 2 hours, 1 ng/ml

| | | |
|---|---|---|
| 1 mg/ml | 23.5 ± 1.2 | 14% |
| 0.1 mg/ml | 21.8 ± 0.8 | 20% |
| 0.001 mg/ml | 25.4 ± 3.6 | / |
| 1 μM indomethacin | 22.6 ± 3.4 | 17% |

PMA stimulation, 2 hours, 10 ng/ml

| | | |
|---|---|---|
| 1 mg/ml | 27.5 ± 4.9 | 18% |
| 0.1 mg/ml | 27.4 ± 4.6 | 18% |
| 0.001 mg/ml | 29.4 ± 4.4 | 12% |
| 1 μM indomethacin | 20.9 ± 4.9 | 37% |

PMA (phorbol-12-myristate-13-acetate) is an activator of protein kinase C. It is a non-physiological activator which mimics the effect of cytokines, including IL-1β on NHK, and encourages the cell to release a lipid molecule, the prostaglandin 6-ketoPGF_{1α}. This prostaglandin is assayed by means of an ELISA. The two activators IL-1β and PMA are used. Indomethacin, an aspirin-type analgesic, is an anti-inflammatory drug (NSAID) which stimulates the reincorporation of free arachidonic acid in triglycerides to decrease the release of eicosanoids. It is used to inhibit the release of 6-ketoPGF_{1α}.

Two experiments were carried out. The first consists of pretreating the NHK with oligorhamnosides for 4 hours and then monitoring the stimulation for 2 hours in the presence of oligorhamnosides. The results are summarized in Table 6 above.

A second experiment was carried out in which the pretreatment with oligorhamnosides was extended to 12 hours. The results are summarized in Table 7 below.

TABLE 7
Results of treatment of NHK with IL-1β and
various oligorhamnoside concentrations after 12
hours of pretreatment with oligorhamnosides.

| | Mean 6-ketoPGF_{1α} | |
|---|---|---|
| Treatment | (pg/ml/μg proteins) | % inhibition |
| Negative control | 19.5 ± 4.5 | |
| Positive control | 35.5 ± 5.3 | |
| (IL-1β 1 ng/ml) | | |

IL-1β stimulation, 2 hours, 1 ng/ml

| | | |
|---|---|---|
| 2 mg/ml | 28.2 ± 3.8 | 21% |
| 1 mg/ml | 30.6 ± 3.1 | 14% |
| 0.5 mg/ml | 35.2 ± 0.4 | / |
| 0.1 mg/ml | 31 ± 5.1 | / |
| Indomethacin | 22.6 ± 3.4 | 36% |

Under these experimental conditions, the release of the mediator 6-ketoPGF_{1α} by the stimulated NHK is low. This appears slightly inhibited, however, when the cells are treated by oligorhamnosides at a high concentration, greater than 1 mg/ml.

Assay of PGE₂ Released by NHK Stimulated by PMA.

Oligorhamnosides were evaluated as an inhibiter of the release of PGE₂ in cellular supernatants. These products were placed in the presence of the NHK at the same time as the PMA at 1 ng/ml. Each condition tested was evaluated for stimulation on 4 wells of NHK.

The results summarized in Table 8 below represent the mean PGE₂ concentration values (pg/ml), after 24 hours of treatment, given in each of the cellular supernatants, stimulated or not, and reported in a quantity of cells expressed in μg.

TABLE 8
Percentage of inhibition of PGE₂ release as a function
of the concentration of rhamnosylated derivatives.

| | 2 mg/ml | 1.5 mg/ml | 1 mg/ml | 0.5 mg/ml | 0.1 mg/ml |
|---|---|---|---|---|---|
| Oligo- | | | 25% | | |
| rhamnosides | 58% | | | | |
| | | 60% | 53% | 27% | / |
| | 72% | | | | |
| | | | 49% | | |

The oligorhamnosides inhibit the release of PGE₂ at rather high concentrations: 2 mg/ml to 1 mg/ml with a mean inhibition of from 50% to 60%. For lower concentrations, its inhibiting capacity decreases and becomes null to 0.1 mg/ml.

Assay of Molecules LTB₄ and PGE₂ Released by Human Neutrophilic Leukocytes.

Neutrophils are isolated from normal human blood and purified by an original method. They are then activated by a stimulation buffer which causes the release of two types of lipid molecules, arachidonic acid derivatives, arising from the cyclooxygenase and lipoxygenase pathways, namely the leukotriene LTB₄ and the prostaglandin PGE₂. This stimulation buffer contains in particular Ca²⁺ and Mg²⁺ ions. These assays are carried out by means of an ELISA-type commercial assay kit.

The cellular viability of the neutrophils treated with oligorhamnosides is evaluated with trypan blue. The viabilities are satisfactory even at the highest oligorhamnoside concentrations.

The results represent the means of the values assayed in two supernatants of cells stimulated during the same experiment.

The results obtained for the inhibition of the release of lipid molecules are summarized in Table 9 below.

TABLE 9
Results of treatment of neutrophilic leukocytes with stimulation
buffer and various oligorhamnoside concentrations.
PGE₂ released (pg/ml)

| Treatment | Without stimulation | Stimulation | % inhibition |
|---|---|---|---|
| Negative control | 3.2 ± 0.3 | 22.1 ± 2.8 | |
| 1 mg/ml | 4 ± 0.7 | 19.6 ± 2.3 | 11% |
| 0.5 mg/ml | 4.3 ± 0.6 | 22.8 ± 0.9 | / |
| 0.1 mg/ml | 4.6 ± 0.6 | 20 ± 2.6 | / |
| 1 μM indomethacin | 4.5 ± 0 | 6.8 ± 1 | 69% |

LTB₄ released (pg/ml)

| | | | |
|---|---|---|---|
| Negative control | 24.3 | 2494 ± 105 | |
| 1 mg/ml | 19.8 | 1857 ± 97 | 26% |
| 0.5 mg/ml | 33.3 | 2199 ± 122 | 12% |
| 0.1 mg/ml | 34.1 | 2073 ± 169 | 17% |
| 0.1 μM NDGA | / | 684 ± 185 | 73% |

Indomethacin is used as a specific inhibiter of the release of PGE₂ and nordihydroguaiaretic acid as a specific inhibiter of the release of LTB₄.

This table makes it possible to conclude that there is a very weak inhibition of the release of PGE₂ (11%) observed after the treatment of the neutrophils with oligorhamnosides. However, in the presence of oligorhamnosides at a concentration of 1 mg/ml, a 26% inhibition of the release of LTB₄ is obtained and confirmed.

In conclusion, oligorhamnosides at a concentration of 1 mg/ml ensure a 27% to 28% inhibition in the release of IL-8 by NHK in two independent experiments, however the release of 6-ketoPGF_{1α} by NHK is very slightly decreased. At higher concentrations of 1 mg/ml to 2 mg/ml, oligorhamnosides inhibit the release of PGE₂ by NHK by 50% to 60% on average. These same molecules at a concentration of 1 mg/ml contribute to an inhibition of the release of LTB₄ by neutrophils by 21% and 26% in two independent experiments. These molecules have no effect on the release of PGE₂ by human neutrophils.

1. A method for the preparation of oligorhamnosides, wherein it comprises the following successive steps:
a) self-condensation of rhamnose in a single reaction step in acetonitrile in the presence of an acid catalyst and precipitation of the oligorhamnosides thus formed; then
b) recovery by filtration of the precipitate obtained following step a) comprising the oligorhamnosides.

2. The method according to claim 1, wherein the temperature of the reaction mixture, during step a), lies between 20° C. and 120° C.

3. The method according to claim 1, wherein the acid catalyst is chosen from the group comprised of hydrochloric acid, sulfuric acid, phosphoric acid, ortho-, meta- and para-toluenesulfonic acid, benzene-sulphonic acid, substituted benzene-sulphonic acids, methane-sulphonic acid, Lewis acids, clay acids, synthetic resin acids, zeolites and combinations thereof.

4. The method according to claim 1, wherein the water formed during the self-condensation reaction of step a) is eliminated physically or chemically.

5. The method according to claim 4, wherein the water elimination technique comprises the use of a desiccation agent chosen among the group consisting of the carbonates, the sulfates, calcium chloride, phosphorus pentoxide, the molecular sieves or combinations of these various desiccation agents.

6. The method according to claim 1, wherein step a) is carried out at atmospheric pressure and under atmosphere of an inert gas.

7. The method according to claim 1, wherein step a) is carried out at reduced pressure, in an autoclave.

8. The method according to claim 1, wherein prior to step b), the reaction mixture is cooled to a temperature in the range between the condensation reaction temperature and 0° C.

9. The method according to claim 8, wherein the reaction mixture is cooled to ambient temperature.

10. The method according to claim 1, wherein the precipitate recovered following step b) is washed with acetonitrile.

11. The method according to claim 1, wherein the acetonitrile included in the filtrate obtained following step b) is evaporated in order to recover a second precipitate containing oligorhamnosides.

12. A composition comprising a mixture of oligorhamnosides obtainable by a method according to claim 1, wherein the aforementioned oligorhamnosides contain from 2 to 12 rhamnose motifs.

13. The composition according to claim 12, wherein the distribution of oligorhamnosides as a function of their degree of polymerization roughly follows a Poisson distribution.

14. The composition according to claim 12, wherein the rhamnose motifs have up to three of their hydroxyl functions implicated in the formation of glycosidic bonds.

15. A medicament containing a composition as defined in claim 12.

16-23. (canceled)

24. The medicament according to claim 15, wherein it contains from 0.001% to 50% by weight of oligorhamnosides.

25. A method for the treatment of skin and/or mucous membranes that are sensitive, irritated, intolerant, of an allergic tendency, aged, exhibiting danger signs, exhibiting a disorder of the cutaneous barrier, exhibiting cutaneous redness or exhibiting a non-pathological immunological imbalance related to intrinsic, extrinsic or hormonal aging, wherein it consists of applying to the skin and/or the mucous membranes of a patient in need thereof a composition according to claim 12.

26. The method to slow the natural aging of the skin and/or to prevent the accelerated aging of skin subjected to external attacks, wherein it consists of applying to the skin of a patient in need thereof a composition according to claim 12.

27. The method according to claim 2, wherein the temperature of the reaction mixture, during step a), lies between 35° C. and 75° C.

28. The method according to claim 3, wherein the acid catalyst is a Lewis acid chosen from the group comprised of zinc chloride and ferric chloride.

29. The method according to claim 3, wherein the acid catalyst is a clay acid chosen from the group comprised of montmorillomite K-10.

30. The method according to claim 6, wherein step a) is carried out under atmosphere of argon or nitrogen.

31. The method according to claim 8, wherein the reaction mixture is cooled to 20° C.

32. The composition according to claim 12, wherein said oligorhamnosides contains from 2 to 9 rhamnose motifs.

33. A method for regulate inflammatory mechanisms comprising the administration of the composition according to claim 12 to a patient in need thereof.

34. The method according to claim 33 for the prevention or treatment of allergic, inflammatory or immune reactions or pathologies of the skin and/or mucous membranes.

35. The method according to claim 33 for the inhibition of immune response related to inflammatory stress.

36. The method according to claim 33 to inhibit leukocyte activation, secretion of keratinocytic cytokines, keratinocytic hyperplasia phenomenon, antigen processing by the dendritic cells of the skin, maturation of antigen-presenting cells, and recognition phenomenon between lymphocytes and antigen-presenting cells.

37. The method according to claim 33 for prevention or treatment of diseases chosen from the group comprised of a topic and/or contact eczema, inflammatory dermatoses, irritant dermatitis, acne, autoimmune diseases such as psoriasis, photo-immunosuppression, vitiligo, pityriasis, sclerodermas, rheumatoid arthritis, Crohn's disease and graft rejection.

38. The method according to claim 33 for prevention and treatment of age-related chronic inflammatory problems and their consequences.

39. The method according to claim 33 for prevention or treatment of diseases chosen from the group comprised of anaphylactic sensitivities, pigmentary anomalies of the skin, dermal hypervascularity and inflammatory fissuring.

40. The method according to claim 33 to reduce the allergenic and/or irritant character of a composition or perfume.

41. The method according to claim 26 to prevent photo-induced aging of the skin.
